(19) United States
(12) Patent Application Publication
(10) Pub. No.: US 2019/0374317 A1
(43) Pub. Date: Dec. 12, 2019

(54) FACE SCANNING AND POSITIONING STRUCTURE USED FOR FULL DENTURE

(71) Applicants: WEN-HSIN CHIOU, Taichung City (TW); JHEN-GUO TSAI, Tainan City (TW)

(72) Inventors: WEN-HSIN CHIOU, Taichung City (TW); JHEN-GUO TSAI, Tainan City (TW)

(21) Appl. No.: 16/001,452

(22) Filed: Jun. 6, 2018

(51) Int. Cl. A61C 9/00; A61B 6/14

(52) U.S. Cl. CPC A61C 9/004; A61B 2090/3966; A61B 6/14

(57) ABSTRACT

A face scanning and positioning structure used for a full denture includes an irregularly shaped body, development projections, and concave portions. The irregularly shaped body defines an asymmetrical face portion along an X-axis, an asymmetrical side portion along a Y-axis, and a thickness along a Z-axis. The development projections are arranged on the asymmetrical face portion and the asymmetrical side portion at predetermined positions. The development projections protrude outwardly. The concave portions are arranged on the asymmetrical face portion and the asymmetrical side portion at predetermined positions. The concave portions each have a concave shape. Through the development projections and the concave portions, the irregularly shaped body is in the form of an asymmetrical structure along the X-axis, the Y-axis, and the Z-axis. Through the irregular protrusions and concave portions, the face scanning and positioning structure can effectively reduce the image overlay error of a specific axis.

### FIELD OF THE INVENTION

The present invention relates to a face scanning and positioning structure, and more particularly to a face scanning and positioning structure used for a full denture.

### BACKGROUND OF THE INVENTION

The demand for making a full denture is common for elderly patients or those who suffer from bad habits (such as smoking, eating betel nut, etc.), resulting in complete loss of teeth. The most common full denture is in the form of a movable denture, which usually includes a whole set of maxillary teeth and mandibular teeth. Before making a full denture or a denture restoration design, it is necessary to record the dynamic intermaxillary relationship to ensure the suitability and comfort of the products. Recording the dynamic intermaxillary relationship for a patient without any tooth is to record the so-called Gothic arch movement path. After the dynamic intermaxillary relationship is recorded, when a 3D scanner is used to scan the patient's face, it is necessary to provide a positioning plate outside the mouth as a positioning feature because the patient has no tooth. However, the conventional positioning plate is generally a standard planar thin plate. The surface of the positioning plate has a black point as a feature. Due to the coincidence of the 3D image after scanning, the positioning error of the Y-axis is large. The design of the planar black point is easy to present an incomplete image for the triangular mesh point 3D image, which affects the accuracy and quality of the denture production.

### SUMMARY OF THE INVENTION

The primary object of the present invention is to provide a face scanning and positioning structure used for a full denture. The face scanning and positioning structure can effectively reduce the image overlay error of a specific axis. It is easy to observe the image overlay error of each axis. The applicability and quality of the denture products can be improved.

In order to achieve the aforesaid object, the present invention provides a face scanning and positioning structure used for a full denture. The face scanning and positioning structure is cooperatively installed to a universal intermaxillary relationship recording device. The face scanning and positioning structure comprises an irregularly shaped body, a plurality of development projections, and a plurality of concave portions.

The irregularly shaped body defines an asymmetrical face portion along an X-axis, an asymmetrical side portion along a Y-axis, and a thickness along a Z-axis. The asymmetrical face portion and the asymmetrical side portion are in the form of an asymmetrical structure. One side of the asymmetrical face portion is provided with a mounting portion. The mounting portion is detachably connected to the universal intermaxillary recording device.

The plurality of development projections are arranged on the asymmetrical face portion and the asymmetrical side portion at predetermined positions. The development projections protrude outwardly.

The plurality of concave portions are arranged on the asymmetrical face portion and the asymmetrical side portion at predetermined positions. The concave portions each have a concave shape.

Wherein, through the development projections and the concave portions, the irregularly shaped body is in the form of an asymmetrical structure along the X-axis, the Y-axis, and the Z-axis.

### BRIEF DESCRIPTION OF THE DRAWINGS

FIG. 1 is a perspective view of the present invention;

FIG. 2 is a schematic view of the present invention when in use; and

FIG. 3 is another schematic view of the present invention when in use.

### DETAILED DESCRIPTION OF THE PREFERRED EMBODIMENTS

Embodiments of the present invention will now be described, by way of example only, with reference to the accompanying drawings.

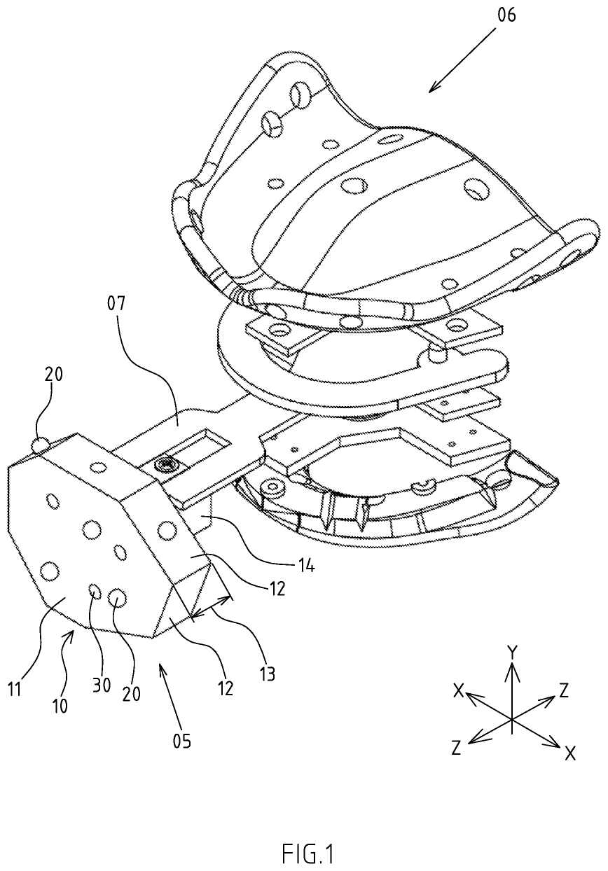

Firstly, referring to FIG. 1 to FIG. 3, the present invention provides a face scanning and positioning structure 05 used for a full denture. The face scanning and positioning structure 05 is cooperatively installed to a universal intermaxillary relationship recording device 06. The face scanning and positioning structure 05 comprises an irregularly shaped body 10, a plurality of development projections 20, and a plurality of concave portions 30.

The irregularly shaped body 10 defines an asymmetrical face portion 11 along an X-axis (only shown in FIG. 1), an asymmetrical side portion 12 along a Y-axis (only shown in FIG. 1), and a thickness 13 along a Z-axis (only shown in FIG. 1). The asymmetrical face portion 11 and the asymmetrical side portion 12 are in the form of an asymmetrical structure. One side of the asymmetrical face portion 11 is provided with a mounting portion 14. The mounting portion 14 is detachably connected to the universal intermaxillary recording device 06. In a specific implementation, the mounting portion 14 is in the form of a slide rail, and is slidably and detachably connected to a coupling portion 07 of the universal intermaxillary recording device 06.

The plurality of development projections 20 are arranged on the asymmetrical face portion 11 and the asymmetrical side portion 12 at predetermined positions. The development projections 20 protrude outwardly.

The plurality of concave portions 30 are arranged on the asymmetrical face portion 11 and the asymmetrical side portion 12 at predetermined positions. The concave portions 30 each have a concave shape.

Wherein, through the development projections 20 and the concave portions 30, the irregularly shaped body 10 is in the form of an asymmetrical structure along the X-axis, the Y-axis, and the Z-axis.

The main specific embodiment and effects of the present invention are described below. Please refer to FIG. 1 to FIG. 3.

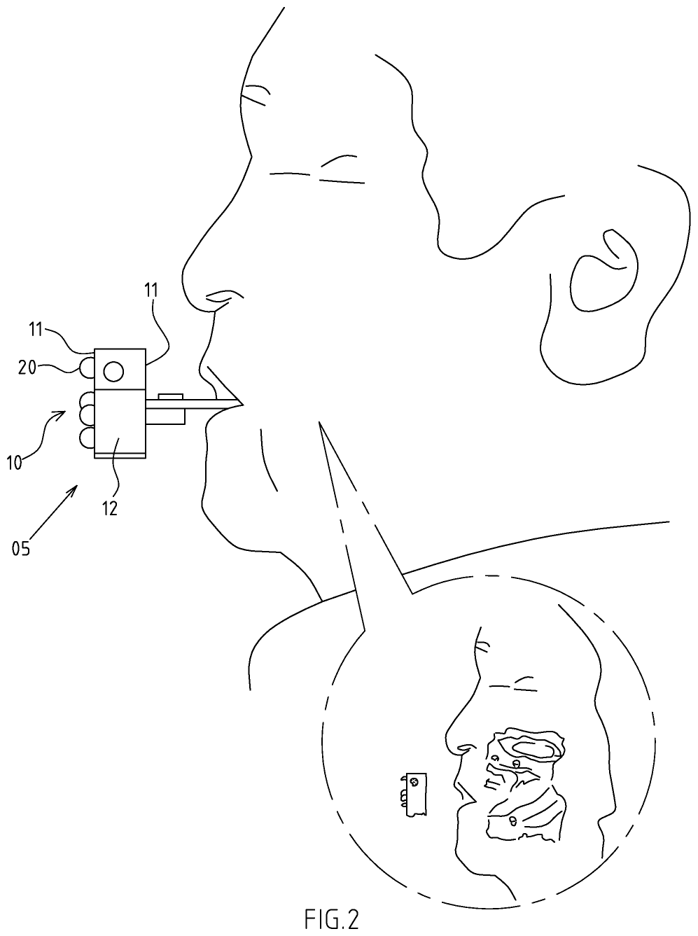

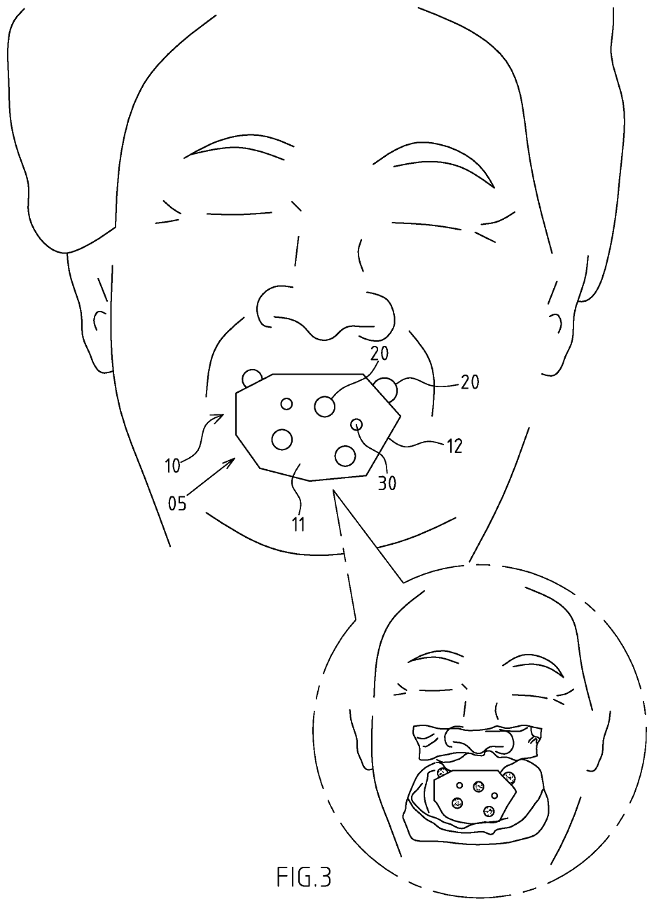

Two sides of the asymmetrical side portion 12 of the present invention are inclined planes of different slopes. In addition, upper and lower sides of the asymmetrical face portion 11 are inclined planes of different slopes. It is worth noting that the development projections 20 of the present invention are embedded development beads made of a barium sulfate material. When an edental patient bites the universal intermaxillary recording device 06 for performing computed tomography scan (i.e., the so-called CT scan is based on the development of bone tissue, as indicated in the lower right of FIG. 2 and FIG. 3), the development projections 20 made of the barium sulfate material generate light spots along the X-axis, the Y-axis, and the Z-axis as feature points for positioning. When a 3D scanner scans the feature points (the triangular mesh point image coincides with the image of the CT scan to obtain an accurate oral position structure, the scan image is not shown in the figures), the positions of the light spots of the development projections 20 and the CT image scan are the same. Therefore, the face scanning and positioning structure 05 of the present invention uses the asymmetrical shape and increases the distance between the axes to generate easily recognizable features during scanning, so as to effectively reduce the image overlay error of a specific axis (such as, the Z-axis, but not limited thereto.) It is easy to observe the image overlay error of each axis.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A face scanning and positioning structure used for a full denture, the face scanning and positioning structure being cooperatively installed to a universal intermaxillary relationship recording device, the face scanning and positioning structure comprising:
an irregularly shaped body, defining an asymmetrical face portion along an X-axis, an asymmetrical side portion along a Y-axis and a thickness along a Z-axis, the asymmetrical face portion and the asymmetrical side portion being in the form of an asymmetrical structure, one side of the asymmetrical face portion being provided with a mounting portion, the mounting portion being detachably connected to the universal intermaxillary recording device;
a plurality of development projections, arranged on the asymmetrical face portion and the asymmetrical side portion at predetermined positions, the development projections protruding outwardly;
a plurality of concave portions, arranged on the asymmetrical face portion and the asymmetrical side portion at predetermined positions, the concave portions each having a concave shape;
wherein through the development projections and the concave portions, the irregularly shaped body is in the form of an asymmetrical structure along the X-axis, the Y-axis, and the Z-axis.

2. The face scanning and positioning structure as claimed in claim 1, wherein two sides of the asymmetrical side portion are inclined planes of different slopes.

3. The face scanning and positioning structure as claimed in claim 2, wherein upper and lower sides of the asymmetrical face portion are inclined planes of different slopes.

4. The face scanning and positioning structure as claimed in claim 1, wherein the development projections are embedded development beads made of a barium sulfate material.

5. The face scanning and positioning structure as claimed in claim 1, wherein the mounting portion is in the form of a slide rail and is slidably and detachably connected to a coupling portion of the universal intermaxillary recording device.
